(19) United States
(12) Patent Application Publication
(10) Pub. No.: US 2026/0207291 A1
(43) Pub. Date: Jul. 23, 2026

(54) MEDICAL IMAGING APPARATUS, SYSTEM AND METHOD

(71) Applicant: SMARTRAY PTY LTD, South Perth (AU)

(72) Inventor: Nils Oscar Nivbrant, Fremantle (AU)

(73) Assignee: SMARTRAY PTY LTD, South Perth, WA (AU)

(21) Appl. No.: 19/158,146

(22) PCT Filed: Aug. 31, 2023
(86) PCT No.: PCT/AU2023/050842

§ 371 Date: Aug. 20, 2025

(30) Foreign Application Priority Data
Feb. 20, 2023 (AU) 2023900425

(51) Int. Cl. A61B 90/00; A61B 6/00; A61B 6/46; A61B 34/20

(52) U.S. Cl. CPC A61B 90/39; A61B 6/463; A61B 6/5247; A61B 34/20; A61B 2090/3762; A61B 2090/3966; A61B 2090/397; A61B 2090/3983; A61B 2090/3991; A61B 2090/3995

(57) ABSTRACT

Provided is medical imaging apparatus (10) comprising a flexible skin patch (12) comprising an adhesive layer (14) on a lower side (16) thereof, and at least one fiducial marker (18) fast on an upper side (20) of said skin patch (12). The fiducial marker (18) comprises a radiopaque core (22) and an infrared reflective coating (24). In this manner, the skin patch (12) is releasably adherable to skin (26) and said fiducial marker (18) is detectable as a single position in space via both stereoscopic computer vision (28) and computed tomography imaging (30) modalities to enable correlation of virtual and physical space as a navigational aid for an image-guided medical procedure. An associated medical imaging system (32) and medical imaging method are also included.

### TECHNICAL FIELD

This invention relates broadly to medical imaging and related imaging-guided medical procedures, and more specifically to medical imaging apparatus, a medical imaging system, and an associated medical imaging method.

### BACKGROUND ART

The following discussion of the background art is intended to facilitate an understanding of the present invention only. The discussion is not an acknowledgement or admission that any of the material referred to is or was part of the common general knowledge as at the priority date of the application.

Medical imaging is known and is conventionally a process of imaging the interior of a body for clinical analysis and medical intervention, as well as visual representation of the function of some organs or tissues. In general, medical imaging seeks to reveal internal structures hidden by the skin and bones, as well as to diagnose and treat disease.

For example, a computed tomography scan (CT scan) is a medical imaging technique used to obtain detailed internal images of the body. CT scanners generally use a rotating X-ray tube and a row of detectors placed in an opposed gantry to measure X-ray attenuations by different tissues inside the body. Multiple X-ray measurements taken from different angles are then typically processed on a computer using tomographic reconstruction algorithms to produce tomographic or cross-sectional images or ‘virtual slices’ of a body.

Due to the usefulness of medical imaging, image-guided surgery and related medical procedures have been developed. Such image-guided surgery is broadly a surgical procedure where the physician uses tracked surgical instruments in conjunction with preoperative or intraoperative medical images in order to directly or indirectly guide the procedure. Image-guided surgery typically helps surgeons perform safer and less invasive procedures and has become a recognised standard of care in managing disorders including cranial, otorhinolaryngology, spine, orthopaedic, and cardiovascular. For example, CT is routinely used as a guidance tool for many interventional radiology procedures including biopsies, aspirations, drain placements, and thermal ablations, in addition to serving as a diagnostic imaging modality.

Accuracy of navigation during image-guided surgery and related medical procedures is vital to ensure patient safety and complication avoidance and various navigation systems have been developed. Two commonly used metrics to assess the accuracy of a navigation system are root mean square (RMS) and target registration error (TRE). RMS is a calculation of the error between the “theoretical” points selected as fiducial markers or anatomical landmarks and the “ground truth,” from the patient's preoperative imaging, e.g. computed tomography (CT) scan.

Applicant has identified shortcomings in the art of conventional CT-guided procedures in measuring the difference between where the fiducial points or anatomical landmarks truly are in 3-dimensional (3D) space and where such navigational points are predicted to be according to the patient's in-situ or preoperative CT imaging. To align anatomical landmarks with preoperative imaging as closely as possible, conventional procedures are often performed using invasive and repetitive methods resulting in increased risk to patient, unnecessary time expenditure and radiation exposure.

For example, current practices often rely on tracking systems along with bone anchored markers for fusion of virtual space (via an X-ray) and physical space (via the bone-anchored markers), where X-ray scanned anatomical landmarks can be consolidated with the physical markers. Such bone-anchored markers inherently require invasive procedures conducted by surgeons who have bone access through incision, as opposed to the vast number of physicians and radiologists using CT guidance for minimally invasive procedures.

Examples of such conventional practices are described in European patent application no EP 3936079 A1 to Spine Align LLC, which describes various intraoperative alignment assessment practices, i.e. occurring or performed during the course of a surgical operation. These practices are described using a trackable surgical tool including a tool dynamic reference frame. Some embodiments of the invention include an assembly with an arrangement of 3D tracked markers that can be utilized for discrete signalling to an acquisition system. In some embodiments, four tracked markers that make up the dynamic reference frame (DRF), and two tracked stray markers (TSMs) are included in the assembly. In some embodiments, the centre of the assembly can include a rotating shield that can be positioned to cover select TSMs, or none at all. In some embodiments, with the tools geometry known, the acquisition system software can interpret which TSMs are exposed, and based on pre-programmed combinations, the tool is able to communicate discrete messages with the acquisition system. In some embodiments, for example, if a first TSM is covered, this can indicate the system is in a particular state as opposed to if a second TSM is covered, which would indicate another state. In some embodiments, because the tool contains a DRF, its location and pose can be interpreted by a 3D-tracking camera, and the arrangement of covered and uncovered stray markers can then be used for communication particular commands or device states. This approach is used to indicate anatomical reference axes to a user, which can otherwise be difficult to interpret.

This prior art system enables software to interpret localization of exposed regions using a CT scan to initialize a patient prior to intraoperative assessments of spinal alignment, and such intraoperative interpretation of acquired data can be performed with or without the use of fiducial landmarks, such as skin-mountable fiducial markers. In this manner, it is possible to initialize a patient's anatomy by calculating displacement vectors from particular regions of interest to another based on this pre-operative and/or intraoperative initialization of data to represent the location of unexposed regions.

These prior art methods are cumbersome, inaccurate (requiring invasive procedures to improve accuracy via mounting of, for example, bone markers), is computationally intensive to perform, and does not provide immediate feedback should the dynamic reference frame or position of a surgical tool change. The current invention was conceived with these shortcomings in mind with a view to ameliorate such conventional practices.

### SUMMARY OF THE INVENTION

The skilled addressee is to appreciate that reference herein to ‘stereoscopic computer vision’ generally refers to any suitable technology for realising artificial systems able to obtain information from multi-dimensional objects in space, and any suitable configuration that deals with how computers or similar processing systems are able to gain positional information of objects in three-dimensional space, as is conventionally known in the art of computer and visual engineering.

It is also to be appreciated that reference herein to ‘real-time’ is to be understood as meaning an instance of time that may include a delay typically resulting from processing, calculation and/or transmission times inherent in electronic processing systems. These transmission and calculations times, albeit of generally small duration, do introduce some measurable delay, i.e. typically less than a second or within milli-or microseconds, but feedback is provided relatively quickly, practically instantaneously or within substantial ‘real-time’.

According to a first aspect of the invention there is provided medical imaging apparatus comprising:
a flexible skin patch comprising an adhesive layer on a lower side thereof so that said skin patch is releasably adherable to skin, in use; and
at least one fiducial marker fast on an upper side of said skin patch, the fiducial marker comprising:
   i. a radiopaque core detectable via a first imaging modality; and
   ii. an infrared reflective coating detectable via a second imaging modality;
      wherein said fiducial marker is detectable as a shared, single position in space via both first and second imaging modalities to enable real-time correlation of virtual and physical space as a navigational aid for an image-guided medical procedure.

The skilled addressee is to appreciate that the first and second imaging modalities are operative or responsive to different electromagnetic spectra.

In an embodiment, the first imaging modality comprises computed tomography, i.e. CT scan.

In an embodiment, the second imaging modality comprises stereoscopic computer vision.

The skilled addressee is to appreciate that the flexible skin patch generally comprises a relatively small bandage, plaster or skin dressing which is releasably adherable to a patient's skin. Similarly, reference to ‘virtual space’ generally refers to three-dimensional space established via the computed tomography imaging modality, and ‘physical space’ as three-dimensional space established via the stereoscopic computer vision modality.

In an embodiment, the flexible skin patch is manufactured from a woven fabric, a polymer, such as PVC, polyethylene or polyurethane, or latex.

In an embodiment, the adhesive layer comprises an acrylate, such as methacrylate and epoxy diacrylate.

In an embodiment, the fiducial marker is manufactured from a radiopaque material which is also infrared reflective, e.g. a metal such as gold, or the like.

According to a second aspect of the invention there is provided a medical imaging system comprising:
a stereoscopic computer vision system configured to monitor a physical space proximate a patient;
a computed tomography scanner configured to perform a CT scan to produce a virtual space of said patient;
at least one medical imaging apparatus comprising a flexible skin patch with an adhesive layer on a lower side thereof and at least one fiducial marker fast on an upper side thereof, the fiducial marker comprising a radiopaque core with an infrared reflective coating, the imaging apparatus adherable to a surface of the patient;
a processor arranged in signal communication with the stereoscopic computer vision system and computed tomography scanner, said processor configured to correlate the virtual and physical space using the fiducial marker as a single, shared corresponding position; and
a display configured to display such correlated space in real-time as a navigational aid for an image-guided medical procedure.

In an embodiment, the system includes a medical instrument for use in the image-guided medical procedure, said instrument configured for detection by the stereoscopic computer vision system and display on the display within such correlated space, i.e. comprising an infrared reflective coating, or the like.

According to a third aspect of the invention there is provided a medical imaging method comprising the steps of:
adhering at least one medical imaging apparatus to a surface of a patient, the medical imaging apparatus comprising a flexible skin patch with an adhesive layer on a lower side thereof and at least one fiducial marker fast on an upper side thereof, the fiducial marker comprising a radiopaque core with an infrared reflective coating;
performing a CT scan on the patient via a computed tomography scanner to produce a virtual space of said patient;
monitoring a physical space proximate the patient via a stereoscopic computer vision system; and
correlating, via a processor, the virtual and physical space using the fiducial marker as single, corresponding position shared between said virtual and physical space as a real-time navigational aid for an image-guided medical procedure.

In an embodiment, the step of adhering the medical imaging apparatus comprises adhering a plurality of apparatus to the surface of the patient.

In an embodiment, the method includes the step of displaying such correlated space, via a suitable display, as a navigational aid for the image-guided medical procedure.

In an embodiment, the method includes the step of detecting a medical instrument for use in the image-guided surgical procedure via the stereoscopic computer vision system and displaying said instrument within such correlated space.

According to a further aspect of the invention there is provided medical imaging apparatus, a medical imaging system, and an associated medical imaging method, substantially as herein described and/or illustrated.

### BRIEF DESCRIPTION OF THE DRAWINGS

The description will be made with reference to the accompanying drawings in which:

FIG. 1 is a diagrammatic perspective-view representation of one embodiment of medical imaging apparatus, in accordance with an aspect of the present invention;

FIG. 2 is a diagrammatic side-sectional representation of the medical imaging apparatus of FIG. 1;

FIG. 3 is diagrammatic perspective-view representation of a medical imaging system, in accordance with an aspect of the present invention; and

FIG. 4 is a diagrammatic top-view representation of an example of one embodiment of medical imaging apparatus applied to the skin of a patient.

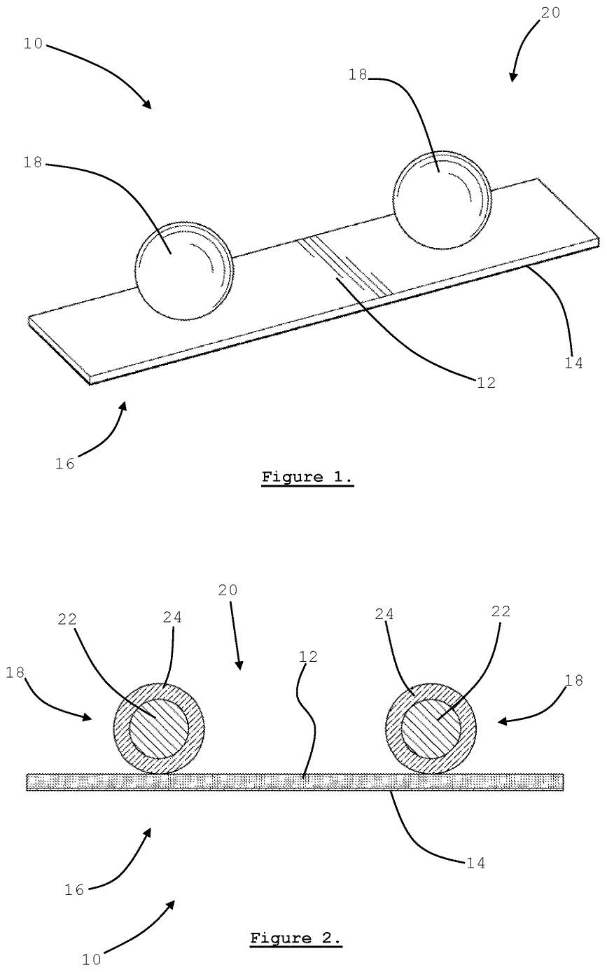

### DETAILED DESCRIPTION OF EMBODIMENTS

Further features of the present invention are more fully described in the following description of several non-limiting embodiments thereof. This description is included solely for the purposes of exemplifying the present invention to the skilled addressee. It should not be understood as a restriction on the broad summary, disclosure or description of the invention as set out above.

In the figures, incorporated to illustrate features of the example embodiment or embodiments, like reference numerals are used to identify like parts throughout. Additionally, features, mechanisms and aspects well-known and understood in the art will not be described in detail, as such features, mechanisms and aspects will be within the understanding of the skilled addressee.

Additionally, the accompanying figures do not represent engineering or design drawings, but provide a functional overview of the invention only. As a result, features and practical construction details required for various embodiments may not be indicated in each figure, but such construction requirements will be within the understanding of the skilled addressee.

Broadly, the present invention provides for medical imaging apparatus, a medical imaging system and an associated medical imaging method to facilitate non-invasive correlation or alignment of anatomical landmarks with preoperative and/or in-situ imaging when performing image-guided surgical or related medical procedures.

With reference now to the accompanying figures, there is shown one possible embodiment of such a medical imaging apparatus 10. Apparatus 10 broadly comprises a flexible skin patch 12 which comprises an adhesive layer 14 on a lower or one side 16 thereof, and at least one fiducial marker 18 fast on an upper or opposite side 20 of the skin patch 12. The flexible skin patch 12 is generally a relatively small bandage, plaster or skin dressing which is releasably adherable to skin 26 of a patient 34. Accordingly, the skin patch 12 may take a variety of shapes and dimensions according to requirements, such variations which are expressly included herein.

In one embodiment, the flexible skin patch 12 is manufactured from a woven fabric, a polymer, such as PVC, polyethylene or polyurethane, or latex. In an embodiment, the adhesive layer 14 comprises an acrylate, such as methacrylate and epoxy diacrylate. Of course, variations hereon are possible and expected. The skin patch 12 may also comprise further layers and/or coatings, such as fluid-impervious or fluid-resistant coatings, or the like.

Importantly, as shown more clearly in FIG. 2, the fiducial marker 18 broadly comprises a radiopaque core 22 and an infrared reflective coating or covering 24, as shown. The radiopaque core is detectable via a first imaging modality, and the infrared reflective coating is detectable via a second imaging modality, said first and second imaging modalities operative in different electromagnetic spectra. For example, in a typical embodiment, the first imaging modality comprises computed tomography, i.e. CT scan, and the second imaging modality comprises stereoscopic computer vision, or the like. However, as the fiducial marker 18 is a single object in space detectable via different imaging modalities, said marker 18 can function as a single position or point shared between virtual and physical space, where virtual space may be defined via a CT scan able to reveal internal anatomical features, and the physical space as visually-identifiable external anatomical features readily visible to a medical practitioner.

The skilled addressee is to appreciate that different embodiments of apparatus 10 may include a different number of fiducial markers 18 fast with the skin patch 12. Similarly, a plurality of fiducial markers 18 on a single skin patch 12 may be arranged in a predetermined orientation and/or relative position with each other, or the like, e.g. in a known triangular orientation, a known number of markers 18 arranged in a line with known separation between them, etc. In one embodiment, the medical imaging apparatus 10 comprises an array of fiducial markers 18 arranged in a predetermined manner.

The radiopaque core 22 may take a variety of forms, as is generally understood in the art. For example, a radiodense material may be used with suitable radio opacity suited to a desired spectra to be easily detectable on x-ray, without obscuring internal organs or producing too severe a shadow in an x-ray imaging modality. As such, radiopaque should not be interpreted as absolute, but including ‘a degree of’ opacity to electromagnetic radiation in a desired spectrum.

Similarly, the reflective coating or covering 24, which may also be configured to be reflective in other frequency spectra depending on requirements, may be realised in a number of ways. For example, a reflective coating comprising suitable pigments and/or spectra-suitable reflective formulations may be applied over the radiopaque core 22, e. g. visible light spectrum, or the like. Alternatively, in one embodiment, the fiducial marker 18 may be manufactured from a radiopaque material which is also infrared or other spectra reflective, a metal such as gold, or the like.

It is further to be appreciated that the fiducial marker 18 may take different forms. For example, any manner of radiopaque core 22 with reflective coating or covering 24 are apposite, as long as the fiducial marker 18 forms a single, shared position or point in space detectable with first and second imaging modalities, as described herein. Accordingly, the fiducial marker 18 is not limited in shape nor material used.

In this manner, the skin patch 12 is releasably adherable to skin 26 of a patient 24 and the fiducial marker(s) 18 is detectable as a single position or point in space via both stereoscopic computer vision 28 and computed tomography 30 imaging modalities to enable correlation of virtual and physical space as a navigational aid for an image-guided surgical procedure. As described, such virtual space generally refers to three-dimensional space established via the computed tomography, i.e. CT scan, imaging modality, and said physical space as three-dimensional space established via the stereoscopic computer vision 28 modality. For example, in the manner described, the fiducial marker(s) 18 is detectable as a single position or point in space as the radiopaque core 22 and coating 24 are generally concentrically arranged spheres with a common centroid occupying a single position in space.

In one embodiment, the fiducial marker 18 is detectable as a shared, single position in space by having a shared or corresponding centre for each of the spherical layers, i.e. the core 22 and coating 24. One example of detecting such a shared, single position in space is via respective detection by means of the first and second imaging modalities, as described, and a suitable image processing computing system with software instructions configured to consider such a shared centre position as an in a Cartesian coordinate system, or the like. For example, such software may consider each respective component of the dual-detected fiducial marker, i.e. core 22 and coating 24, as a circle or sphere which can be juxtaposed or overlaid to determine a centre point or position, or at least common portion which can function as an origin, or the like. Variations hereon are possible and expected and expressly included within the present disclosure.

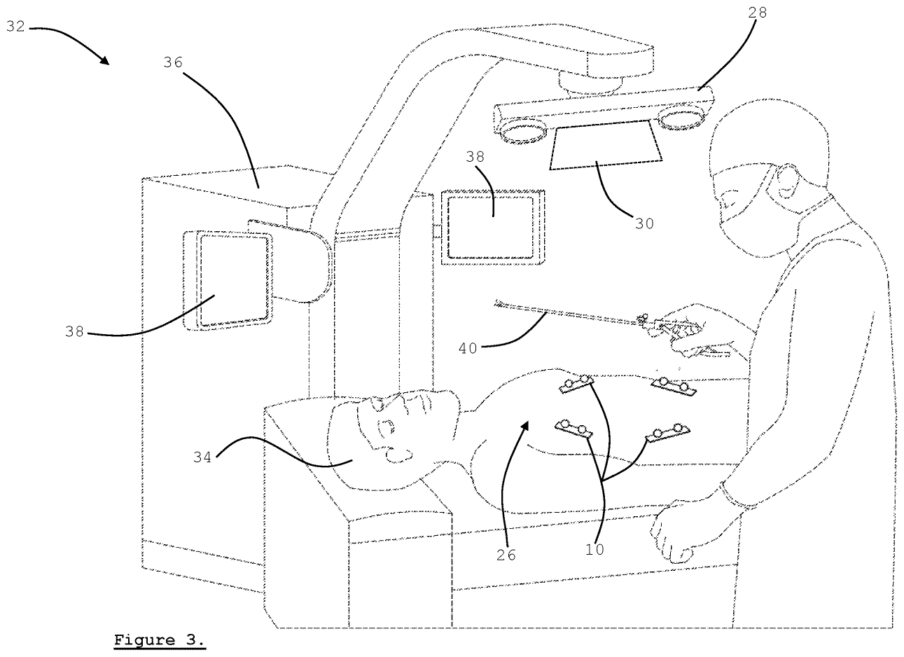

The present invention further includes an associated medical imaging system 32, one possible embodiment of which is exemplified in FIG. 3. Such a system 32 broadly includes a stereoscopic computer vision system 28 which is configured to monitor a physical space proximate a patient 34, as well as a computed tomography scanner 30 which is configured to perform a CT scan to produce a virtual space of said patient 34. Such stereoscopic computer vision system 28 and computed tomography scanner 30 are known in the art and will not be described in detail.

System 32 also operatively includes at least one medical imaging apparatus 10, as described above, which is adherable to a surface, typically skin 26, of the patient 34, as required. System 32 further includes a suitable processor 36 which is arranged in signal communication with the stereoscopic computer vision system 28 and computed tomography scanner 30, with the processor 36 configured to correlate the virtual and physical space using the fiducial marker(s) 18 as corresponding point. In this manner, as described, system 32 is able to correlate or align anatomical landmarks in the physical space with preoperative imaging in the virtual space.

System 32 also generally includes a suitable display 38 which is arranged in signal communication with the processor 26 and which is configured to display such correlated space as a navigational aid for an image-guided medical procedure. Accordingly, system 32 is able to display alignment of the virtual and physical space, often in real-time, in order to guide a physician when performing a medical procedure.

Typically, the system 32 includes a medical instrument 40 for use in the image-guided medical procedure, said instrument comprising an infrared (or other spectra) reflective coating for detection by the stereoscopic computer vision system 28 and display on the display 38 within such correlated space. The medical instrument 40 may also be detectable by the computed tomography scanner 30 and displayed on the display 38 within such correlated space, i.e. alignment between physical and virtual space.

The skilled addressee is to appreciate that the present invention further includes an associated medical imaging method comprising the steps of adhering at least one medical imaging apparatus 10 to a surface of a patient 34, performing a CT scan on the patient via a computed tomography scanner 30 to produce a virtual space of said patient, monitoring a physical space proximate the patient via a stereoscopic computer vision system 28, and correlating, via a processor 36, the virtual and physical space using the fiducial marker(s) 18 as corresponding point as a navigational aid for an image-guided medical procedure.

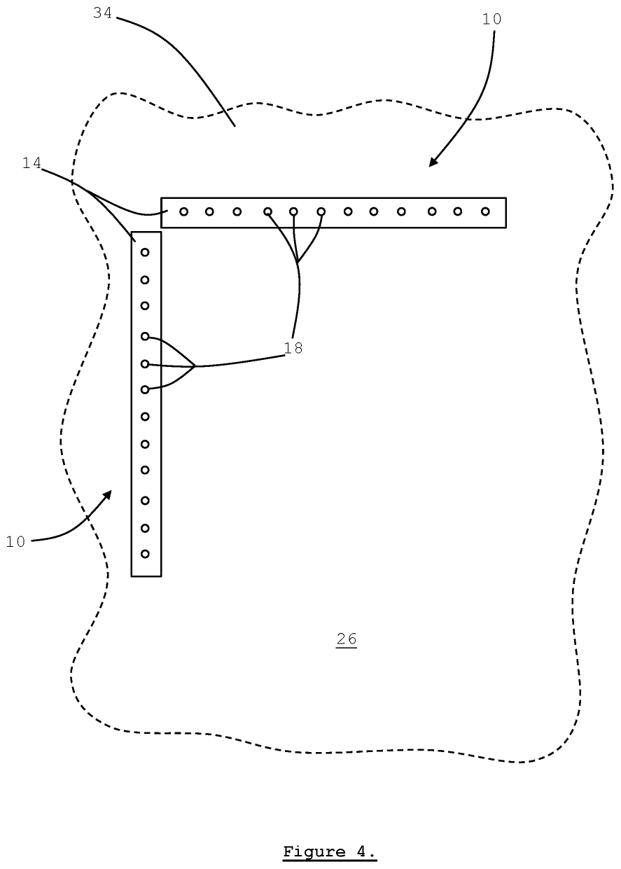

In an embodiment, the step of adhering the medical imaging apparatus 10 comprises adhering a plurality of apparatus 10 to the surface of the patient 34. In one embodiment, a plurality of imaging apparatus 10 is adhered to a patient 34 in a predetermined or desired manner, depending on a medical procedure to be performed. For example, as shown in FIG. 4, in one embodiment the adhesive layer 14 may comprise a strip having a plurality of fiducial markers 18, e.g. 10, arranged therealong, with two such strips 14 adhered perpendicular to each other on the patient, i.e. representing an x/y graph of markers 18 where desired.

Typically, the method includes the step of displaying such correlated space, via a suitable display 38, as a navigational aid for the image-guided medical procedure. Additionally, the method typically includes the step of detecting a medical instrument 40 for use in the image-guided medical procedure via the stereoscopic computer vision system 28 as well as the computed tomography scanner 30 and displaying said instrument within such correlated space.

Applicant believes it particularly advantageous that the present invention provides for means to facilitate a reduction in an overall duration of a surgical or related medical procedure via improving accuracy and precision in applying a medical instrument 40 to a desired location within a patient, as well an associated reduction in radiation exposure due to fewer CT passes required when assessing position during such an image-guided surgical or related medical procedure. Similarly, by having the fiducial marker(s) 18 detectable via different imaging modalities but occupying a single shared position in space, facilitates correlation between virtual and physical space without requiring intensive computational power, which also facilitates real-time display of such correlation as a navigational aid for an image-guided medical procedure.

As a result, via a simple application of medical imaging apparatus 10 to a patient, accurate and real-time medical guidance is achieved when performing minor surgical procedures, such as injections, or the like, without requiring various X-ray or similar scans of the patient, nor the use of elaborate surgical tables with alignment structures, nor patient positioning structures. As a result, both patient and medical practitioner are afforded some freedom of movement during minor medical procedures, such as injections, without adverse effects, resulting in increased patient comfort.

Optional embodiments of the present invention may also be said to broadly consist in the parts, elements and features referred to or indicated herein, individually or collectively, in any or all combinations of two or more of the parts, elements or features, and wherein specific integers are mentioned herein which have known equivalents in the art to which the invention relates, such known equivalents are deemed to be incorporated herein as if individually set forth. In the example embodiments, well-known processes, well-known device structures, and well-known technologies are not described in detail, as such will be readily understood by the skilled addressee.

The use of the terms “a”, “an”, “said”, “the”, and/or similar referents in the context of describing various embodiments (especially in the context of the claimed subject matter) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to, ”) unless otherwise noted. As used herein, the term “and/or” includes any and all combinations of one or more of the associated listed items.

It is to be appreciated that reference to “one example” or “an example” of the invention, or similar exemplary language (e.g., “such as”) herein, is not made in an exclusive sense. Accordingly, one example may exemplify certain aspects of the invention, whilst other aspects are exemplified in a different example. These examples are intended to assist the skilled person in performing the invention and are not intended to limit the overall scope of the invention in any way unless the context clearly indicates otherwise.

Variations (e.g. modifications and/or enhancements) of one or more embodiments described herein might become apparent to those of ordinary skill in the art upon reading this application. The inventor(s) expects skilled artisans to employ such variations as appropriate, and the inventor(s) intends for the claimed subject matter to be practiced other than as specifically described herein.

Any method steps, processes, and operations described herein are not to be construed as necessarily requiring their performance in the particular order discussed or illustrated, unless specifically identified as an order of performance. It is also to be understood that additional or alternative steps may be employed.

1-12. (canceled)

13. A medical imaging apparatus comprising:
a flexible skin patch comprising an adhesive layer on a lower side thereof so that said skin patch is releasably adherable to skin, in use; and
at least one fiducial marker fast on an upper side of said skin patch, the fiducial marker comprising:
i. a radiopaque core detectable via a first imaging modality; and
ii. an infrared reflective coating detectable via a second imaging modality;
wherein said fiducial marker is detectable as a shared, single position in space via both first and second imaging modalities, and wherein a plurality of such fiducial markers is adherable to skin of a patient, by means of said skin patch(es), to define an array of fiducial markers in a Cartesian coordinate system to enable real-time correlation of virtual and physical space by using the fiducial markers in said array each as a single, shared corresponding position within the Cartesian coordinate system as a navigational aid for an image-guided medical procedure.

14. The medical imaging apparatus of claim 13, wherein the first imaging modality comprises computed tomography.

15. The medical imaging apparatus of claim 13, wherein the second imaging modality comprises stereoscopic computer vision.

16. The medical imaging apparatus of claim 13, wherein the flexible skin patch is manufactured from a woven fabric, a polymer, polyethylene, polyurethane, or latex.

17. The medical imaging apparatus of claim 13, wherein the adhesive layer comprises an acrylate, such as methacrylate or epoxy diacrylate.

18. The medical imaging apparatus of claim 13, wherein the fiducial marker is manufactured from a radiopaque material which is also infrared reflective.

19. A medical imaging system comprising:
a stereoscopic computer vision system configured to monitor a physical space proximate a patient;
a computed tomography scanner configured to perform a CT scan to produce a virtual space of said patient;
at least one medical imaging apparatus comprising a flexible skin patch with an adhesive layer on a lower side thereof and at least one fiducial marker fast on an upper side thereof, the fiducial marker comprising a radiopaque core with an infrared reflective coating, wherein a plurality of such fiducial markers is adherable to a surface of the patient, by means of said skin patch(es), to define an array of fiducial markers in a Cartesian coordinate system;
a processor arranged in signal communication with the stereoscopic computer vision system and computed tomography scanner, said processor configured to correlate the virtual and physical space using the fiducial markers in said array each as a single, shared corresponding position within the Cartesian coordinate system; and
a display configured to display such correlated space in real-time as a navigational aid for an image-guided medical procedure.

20. The medical imaging system of claim 19, which includes a medical instrument for use in the image-guided medical procedure, said instrument configured for detection by the stereoscopic computer vision system and display on the display within such correlated space.

21. A medical imaging method comprising the steps of:
adhering a plurality of medical imaging apparatus to a surface of a patient, the medical imaging apparatus comprising a flexible skin patch with an adhesive layer on a lower side thereof and at least one fiducial marker fast on an upper side thereof, the fiducial marker comprising a radiopaque core with an infrared reflective coating, said plurality of fiducial markers defining an array in a Cartesian coordinate system;
performing a CT scan on the patient via a computed tomography scanner to produce a virtual space of said patient;
monitoring a physical space proximate the patient via a stereoscopic computer vision system; and
correlating, via a processor, the virtual and physical space using the array of fiducial markers each as a single, corresponding position shared between said virtual and physical space as a real-time navigational aid for an image-guided medical procedure.

22. The method of claim 21, which includes the step of displaying such correlated space, via a suitable display, in real-time as a navigational aid for the image-guided medical procedure.

23. The method of claim 21, which includes the step of detecting a medical instrument for use in the image-guided surgical procedure via the stereoscopic computer vision system and displaying said instrument within such correlated space.
